(19) United States
(12) Patent Application Publication
(10) Pub. No.: US 2019/0013095 A1
(43) Pub. Date: Jan. 10, 2019

(54) A PATIENT PROCEDURE SCHEDULE THROUGHPUT OPTIMISER SUPERVISED MACHINE LEARNING SYSTEM

(71) Applicant: HRO Holdings Pty Ltd., Heidelberg, VIC (AU)

(72) Inventor: Jock Lawrie, Melbourne, Victoria (AU)

(73) Assignee: HRO Holdings Pty Ltd., Heidelberg, VIC (AU)

(21) Appl. No.: 16/066,597

(22) PCT Filed: Jan. 14, 2017
(86) PCT No.: PCT/AU2017/050028

§ 371 Date: Jun. 27, 2018

(30) Foreign Application Priority Data
Jan. 15, 2016 (AU) 2016900118

(51) Int. Cl. G16H 40/20; G16H 10/60; G06N 7/00; G06N 99/00

(52) U.S. Cl. CPC G16H 40/20; G06N 99/005; G06N 7/005; G16H 10/60

(57) ABSTRACT

There is provided a patient procedure schedule throughput optimiser system comprising: a data extractor module configured for receiving patient procedure training data from a patient procedure schedule data database, a machine learning module having as input the patient procedure training data, the patient procedure training data representing a plurality of prior patient procedures and a duration for each of the prior patient procedures and wherein the machine learning module is configured for training using the patient procedure training data for generating a plurality of patient procedure duration probability distribution models; a trained machine module configured in accordance with the patient procedure duration probability distribution models and having as input schedule data, the schedule data representing a plurality of future patient procedures, and wherein the trained machine module is configured for calculating patient procedure duration probability distributions for each of the future patient procedures; a schedule optimiser module having as input the patient procedure duration probability distributions and wherein the schedule optimiser module is configured for calculating an optimised patient procedure schedule in accordance with the patient procedure duration probability distributions and wherein the schedule optimiser module is configured for optimising the number of sessions of the optimised patient procedure schedule; and a schedule interface configured to display the optimised patient procedure schedule.

### FIELD OF THE INVENTION

The present invention relates to supervised machine learning and optimisation and in particular, supervised machine learning and optimisation of patient procedure sessions schedules.

### BACKGROUND OF THE INVENTION

Surgeons and hospital administrative staff spend considerable time creating and updating patient procedure schedules.

However, given the billions of potential procedure schedule permutations, humans cannot optimise scheduling of procedures.

As such current hospital schedules are sub optimal, resulting in both unnecessary expenditure including in either paying surgeons who are idle or paying surgeons overtime and sub optimal patient throughput. Sub optimal patient throughput results in hospital-induced postponements which are heavily penalised by the state. This problem is ignored by hospitals.

Furthermore, most schedules have additional constraints that must be respected, such as treating patients before a due date or patients who can only attend hospital on certain days due to work commitments. Ultimately these constraints mean aligning patients, staff and other resources such as surgical equipment.

It is to be understood that, if any prior art information is referred to herein, such reference does not constitute an admission that the information forms part of the common general knowledge in the art, in Australia or any other country.

### SUMMARY OF THE DISCLOSURE

There is provided herein a system that uses artificial intelligence/supervised machine learning system select an optimal schedule from potentially billions of potential candidate schedule placement permutations that maximises patient throughput, minimises the number of session required, and optimises schedule completion times.

Specifically, the system that takes a set of patient procedures awaiting session allocation and, utilising supervised machine learning, generates an optimised schedule that optimises patient throughput.

Further, the system utilises a machine learning module which is trained utilising training data taking the form of prior patient procedure training data.

We discovered that certain patient procedures are far “riskier” of not finishing on time as compared to other procedures and therefore designed the present supervised machine learning system (which, in embodiments, may take the form of an artificial neural network (ANN)) such that the machine learning module outputs probability distribution modules representing the probability of a session finishing on time.

These probability distribution modules are then used to configure a trained machine module to calculate patient procedure duration probability distributions for each of a plurality of scheduled patient procedures.

Thereafter, the system then optimises the patient schedule utilising these calculated patient procedure duration probability distributions to optimise the number of sessions of the schedule (patient throughput).

In embodiments, the system optimisation takes into account multivariable and dynamically changing constraints. In yet further embodiments, the optimised patient procedure schedule may be utilised as feedback for further training the machine learning module.

To date, no scheduling system utilises supervised machine learning for the purposes of maximises patient throughput, minimises the number of session required, and optimises schedule completion times, let alone one used for calculating a plurality of patient procedure duration probability distributions which are then optimised utilising an optimiser which optimises sessions from potentially billions of potential permutations.

As such, with the foregoing in mind, in accordance with one aspect, there is provided a patient procedure schedule throughput optimiser system comprising: a data extractor module configured for receiving patient procedure training data from a patient procedure schedule data database, a machine learning module having as input the patient procedure training data, the patient procedure training data representing a plurality of prior patient procedures and a duration for each of the prior patient procedures and wherein the machine learning module is configured for training using the patient procedure training data for generating a plurality of patient procedure duration probability distribution models; a trained machine module configured in accordance with the patient procedure duration probability distribution models and having as input schedule data, the schedule data representing a plurality of future patient procedures, and wherein the trained machine module is configured for calculating patient procedure duration probability distributions for each of the future patient procedures; a schedule optimiser module having as input the patient procedure duration probability distributions and wherein the schedule optimiser module is configured for calculating an optimised patient procedure schedule in accordance with the patient procedure duration probability distributions and wherein the schedule optimiser module is configured for optimising the number of sessions of the optimised patient procedure schedule; and a schedule interface configured to display the optimised patient procedure schedule.

The schedule optimiser module may be configured for calculating the optimised patient procedure schedule using probability distribution variance threshold discrimination of each of the patient procedure duration probability distributions for the plurality of future patient procedures.

The patient procedure training data may further represent a procedure type for each of the prior patient procedures; and

the schedule data may further represent a procedure type for each of the future patient procedures and wherein:

the machine learning module may be configured for generating the session duration probability distribution models further in accordance with the procedure type for each of the prior patient procedures; and the optimiser module may be further configured for generating the throughput optimised schedule further in accordance with the procedure type for each of the future patient procedures.

The patient procedure training data may further represent patient specific information for each of the prior patient procedures; and

the input schedule data may further represent patient specific information for each of the future patient procedures and wherein:

the machine learning module may be configured for generating the session duration probability distribution models further in accordance with the patient specific information for each of the prior patient procedures; and the optimiser module may be further configured for calculating the optimised patient procedure schedule further in accordance with the patient specific information for each of the future patient procedures.

The patient procedure training data may further represent surgeon specific information for each of the prior patient procedures; and

the input schedule data may further represent surgeon specific information for each of the future patient procedures and wherein:

the machine learning module may be configured for generating the session duration probability distribution models further in accordance with the surgeon specific information for each of the prior patient procedures; and

the optimiser module may be further configured for calculating the optimised patient procedure schedule further in accordance with the surgeon specific information for each of the future patient procedures.

The surgeon specific information may includes a surgeon specified session duration estimations.

The trained machine module may be configured for calculating the patient procedure duration probability distributions in accordance with the session duration estimations.

The trained machine module may be configured for calculating the patient procedure duration probability distributions in accordance with a weighting of the session duration estimations.

The weighting may be between 0 and 1.

The trained machine module may be configured for calculating the number of sessions allocated to the surgeon in accordance with the patient procedure training data and calculate the weighting in accordance with the number of sessions.

The schedule optimiser module may be configured for calculating the optimised patient procedure schedule in accordance with constraints.

The constraints may include a constraint of maintaining patients to assigned session.

The constraints may include a constraint of assigning patients to schedules on or before a patient treatment due date.

The constraints may include a prioritising allocation of patients who are already overdue or who are becoming overdue.

The constraints may include a constraint of assigning patients according to patient attendance ability constraints.

The constraints may include a constraint of ensuring patients are allocated for a maximum of one session.

The constraints may include a constraint of allocating surgeons at least a minimum amount of time for sessions that are shared by multiple surgeons.

The patient procedure training data may further represent a procedure type, patient specific information and surgeon specific information for each of the prior patient procedures; and the input schedule data may further represent procedure type, patient specific information and surgeon specific information for each of the future patient procedures and wherein: the machine learning module may be configured for generating the session duration probability distribution models further in accordance with the procedure type, patient specific information and surgeon specific information for each of the prior patient procedures; and the optimiser module may be further configured for calculating the optimised patient procedure schedule further in accordance with the procedure type, patient specific information and surgeon specific information for each of the future patient procedures.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows the data flow of the supervised machine learning task of optimising patient procedure schedules in accordance with an embodiment of the present disclosure;

FIG. 2 shows a system for the supervised machine learning optimisation of patient procedure schedules in accordance with an embodiment of the present disclosure;

FIG. 3 shows an exemplary model/view/controller (MVC) of a patient procedure schedule optimising competing device in accordance with an embodiment of the present disclosure; and

FIG. 4 shows exemplary patient procedure duration probability distributions for differing patient procedures schedules.

### DESCRIPTION OF EMBODIMENTS

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure.

Before the structures, systems and associated methods relating to the patient procedure schedule throughput optimiser supervised machine learning system are disclosed and described, it is to be understood that this disclosure is not limited to the particular configurations, process steps, and materials disclosed herein as such may vary somewhat. It is also to be understood that the terminology employed herein is used for the purpose of describing particular embodiments only and is not intended to be limiting since the scope of the disclosure will be limited only by the claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterised by,” and grammatical equivalents thereof are inclusive or open-ended terms that do not exclude additional, unrecited elements or method steps.

It should be noted in the following description that like or the same reference numerals in different embodiments denote the same or similar features.

### Data Flow

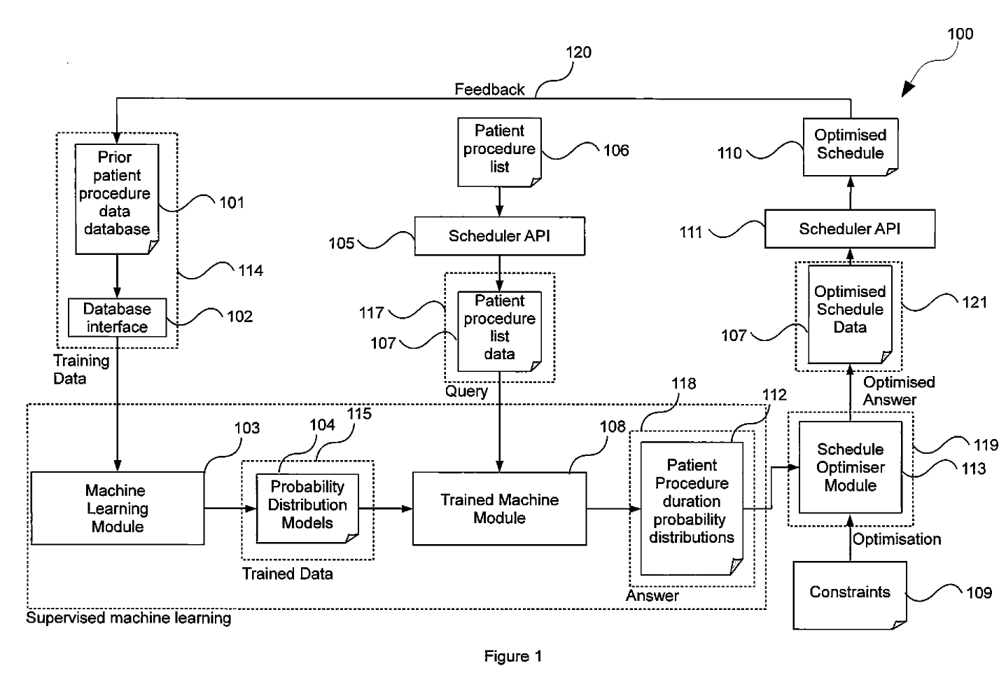

Turning to FIG. 1, there is shown an illustrative data flow 100 of the supervised machine learning and optimisation of patient procedure schedules.

In general terms, the following nomenclature will be utilised:
a. Patient procedure: An individual surgical case. E.g., 25 year old male, non-smoker, non-diabetic who requires a knee reconstruction with Dr Smith. Estimated duration=3 hours.
b. Session: A list of cases. E.g., a session may have 3 cases. Typically the cases will have the same surgeon but not always.
c. Schedule: A set of sessions allocated to specific operating theatres at specific times. E.g., a schedule may contain 2 sessions, one in Theatre 1 on Monday 9th January 8.30-12.30, the other in Theatre 3 on Tuesday 10th January 13.30-17.30.

As alluded to above, the present embodiments utilise an artificial intelligence/supervised machine learning technique wherein a machine learning module is trained utilising training data 114 (being prior patient procedure schedule data) to generate trained data 115 which, in accordance with preferred embodiments represents a plurality of probability distribution models.

As will become apparent from the ensuing description, and again, in general terms, the system generates schedules that are optimised to:
a. Maximise the number of patient procedures completed;
b. Minimise the number of required sessions (e.g surgeons with a short waiting list may not fill all sessions available to him/her, whereas surgeons with a long waiting list will fill all sessions available to him/her); and
c. Generate schedules having sessions with a high chance of finishing on time (i.e. low-risk sessions . . . avoid scheduling sessions having a high chance of finishing late).

The data flow 100 further represents a trained machine module 108 which is configured utilising the trained data 115 (being the plurality of probability distribution models).

In this regard, the configured/trained machine module 108 is configured for receiving a query 117 (being future patient procedures) and outputting an answer 118 (being patient procedure duration probability distributions for each of the sessions).

As such, the probability for each of the schedule sessions finishing on time is ascertained.

Thereafter, the data flow 100 represents optimisation of the patient procedure schedule utilising the patient procedure duration probability distributions to optimise at least one of the number of completed sessions (patient throughput).

In this way, an optimised patient procedures schedule is calculated which may then be utilised to update a schedule in a calendar application or server.

In very general terms the patient procedure schedule data (training data) 101 consists of past patient procedures, together with the observed duration (including anaesthetic) of each patient procedure.

The patient procedure list 106 is the list of sessions for future patient procedures, some of which may already be booked into specific sessions. The observed duration of these future procedures is not yet available.

The machine learning module 103 takes in the training data and outputs a trained data model 115 (the probability distribution models 104). The trained model 108 takes in data of a number of patient procedures and outputs the probability distribution of each of the patient procedures. E.g., a procedure may have a 20% chance of taking 50 mins, 40% chance of taking 60 mins and a 40% chance of taking 70 mins (this is a very simplified example for the purpose of illustration).

Then, the probability distributions 112 of each of the proposed patient procedures is then fed into the schedule optimiser module 113 that, in embodiments:
a. Iterates through all (or almost all) possible patient procedure session placements (permutations) and computes the probability distribution of each session's duration (taking into account the duration probability distributions of the one or more patient procedures assigned to each session). Depending on the length of the waiting list and other constraints (see below), there may be billions of candidate session permutations; and
b. Selects from these possible sessions a subset of sessions that maximises the chance of a schedule finishing on time and minimises the number of sessions, whilst also meeting constraints including:
   i. Patient procedures which are already booked are not moved to another sessions by the optimiser module 113;
   ii. Patients for patient procedures who cannot attend on certain dates (due to work commitments for example) are not scheduled for said dates by the optimiser module 113;
   iii. For patient procedures which are urgent, overdue or close to being overdue are prioritised by the optimiser module 113; and
   iv. Sessions must have less than 20% chance of finishing 45+ mins late. The 20% threshold is configurable and may be altered in the future. For example, as the model improves and becomes more precise, a 10% threshold may be used instead.

Specifically, the data flow 100 shows the machine learning module 103 which is configured for calculating a plurality of probability distribution models in accordance with patient procedure training data 101. As alluded to above, the machine learning module 103 is, in a preferred embodiment, configured for calculating the “risk” of certain types of procedures finishing within the allocated session time.

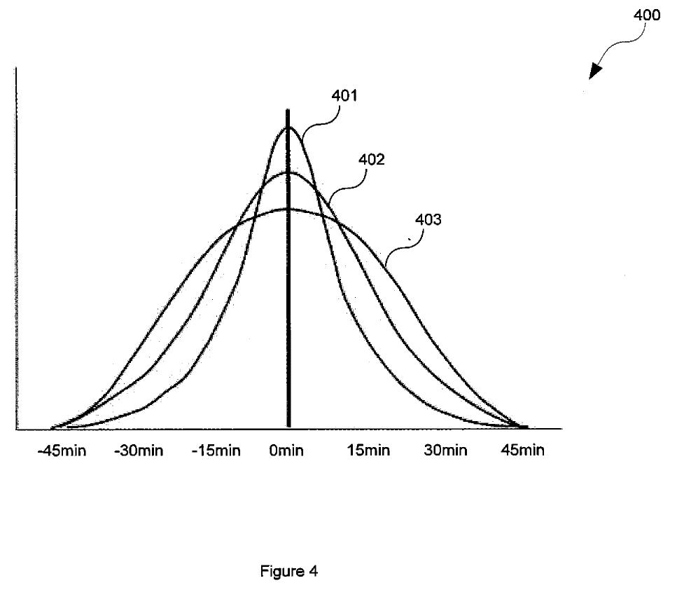

In a preferred embodiment, the machine learning module 103 outputs statistical probability distributions (as a substantially shown in FIG. 4) for each type of procedure which may then be subsequently used by the trained machine module 108 for optimising a procedure schedule.

Specifically, in embodiments, the machine learning module 103 receives prior patient procedure training data 101 via a database interface 102, such as a hospital or clinic database. In other embodiments, the patient procedure training data 101 may be provided to the machine learning module 103 in other manners as opposed to necessarily utilising a database interface 102.

Preferably, the patient procedure training data 101 represents a plurality of prior schedule sessions, each session having an associated one or more procedures, each of which may have a procedure type and an observed or recorded actual duration.

As will be described in further detail below, the machine learning module 103 utilises various data (including one or more of procedure type, surgeon specific information and patient specific information) to calculate the “risk” of particular types of procedures finishing on time.

In a preferred embodiment, the machine learning module 103 utilises statistical probability distributions to represent the “risk” of a proposed procedure finishing on time.

Specifically, turning to FIG. 4, there are shown three different types of procedures 401-403 each having differing risk profiles of finishing on time as is represented by the respective probability distribution curve.

Specifically, as can be seen, procedure 401 has a much greater probability of finishing on time as compared to procedure 403. For example, procedure 401 may represent a routine patient procedure, such as the taking of a blood sample or the like which is more likely to finish on time given the routine nature of the procedure as, for example, compared to procedure 403 which may be a more complex medical procedure, such as removal of an appendix or the like which has a higher probability of not finishing on time given potential complexities that may arise during the procedure.

As alluded to above, the machine learning module 103 may utilise the procedure type in order to calculate the appropriate probability distributions. In the embodiment shown in FIG. 4, the X axis shows probability distributions of the relevant procedure finishing up to 45 minutes early or late.

However, and as alluded to above, the machine learning module 103 may take into account other data as opposed to procedure type including that which is described in further detail below. For example, certain surgeons may be more reliable as compared to others for finishing on time and therefore the machine learning module 103 may additionally take into account a surgeon ID.

In a further embodiment, the machine learning module 103 may take into account patient specific information wherein, for example, elderly patients, as may be evident from the patient procedure training data 101 may experience a greater likelihood of complication as compared to younger patients during surgical procedures.

As can be seen from FIG. 1, the probability distribution models 104 are then utilised to configure a trained machine module 108 so as to be able to receive a query 117 and output an answer 118.

As is represented, the query 117 is patient procedure schedule data representing a plurality of patient procedures and wherein one or more patient procedures are assigned to the available sessions of the schedule and wherein the answer 118 generated by the trained machine module 108 represents a probability distribution of each of the schedule sessions finishing on time.

As can be further seen from FIG. 1, the data flow 100 further comprises an optimiser module 109 which is configured for optimising a schedule 106 in accordance with the probability distributions 104 calculated by the trained machine module 108.

In embodiments, the schedule optimiser module 113 optimises the schedule according to various methods and constraints 109 including 1) Each session having good statistical properties (such as a good chance of finishing within 15 minutes of the session's scheduled finishing time) 2) keeping booked patients in the session to which they have been assigned, 3) ensuring patients are treated on or before their due date, 4) prioritising patients who are already over due, or who are becoming overdue in the next 4 weeks, 5) ensuring patients are only proposed for sessions that they can attend, 6) ensuring patients are proposed for a maximum of one 1 session and 7) ensuring a minimum amount of time per surgeon in sessions that are shared by multiple surgeons.

Specifically, the schedule 106 may represent a future patient procedure schedule involving a plurality of surgeons, patients, procedures, surgical equipment and the like.

As can be seen, the trained machine module 108 is configured for receiving the schedule data 107 from the schedule 106 which, in embodiments may be retrieved via a schedule API 106, so as to retrieve the schedule data from a calendar application or server.

In a preferred embodiment, the schedule data 107 comprises the same or at least a subset of the same data fields of the training data 101. Specifically, in a preferred embodiment, the schedule data 107 also comprises procedure type data representing the type of medical procedure for each session of the schedule so as to allow the trained machine module 108 is to calculate appropriate probability distributions taking into account the procedure type. As alluded to above, the schedule data 107 may additionally comprise other information, such as surgeon specific information, patient specific information and any other information relevant to the duration of the session.

The calculated patient procedure duration probability distributions 112 calculated for each of the sessions is fed into the session optimiser module 113.

In embodiments, certain constraints 109 (including those which are described in further detail below) may be input into the session optimiser module 113 so as to restrict the optimisation of the schedule data by the session optimiser module 113 accordingly.

The session optimiser module 113 iterates through a plurality of session placement permutations, taking into account the relevant probability distributions 112, so as to optimise the number of sessions (patient throughput) of the schedule 106. The session optimiser module 113 may utilise a Monte Carlo permutation, minimising function or other suitable optimisation algorithm for the purposes of calculating the most optimal permutation.

In this way, the trained machine module 108 may potentially test billions of combinations of procedure session placements arriving at the most or near most optimal schedule.

The session optimiser module 113 outputs optimise schedule data 112 which may then be utilised for updating the schedule 106 to generate a throughput optimise schedule 100. Again, the schedule API 111 may be utilised to update the schedule 106 of a calendar application or server.

### System

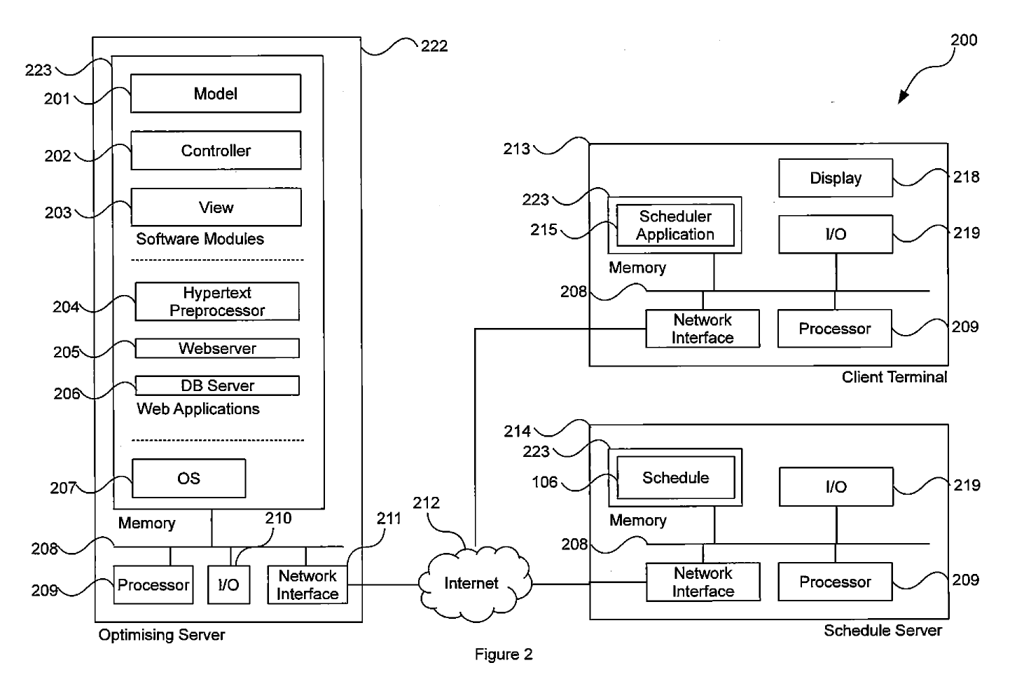

Turning now to FIG. 2, there is shown an exemplary computer system 200 for implementing the various computer processes described herein.

In the embodiment shown, the system 200 take the form of a Web server architecture which, further specifically, take the form of a schedule optimising server 222 in operable communication with at least one client terminal 213 (such as a personal computer device) and a schedule server 214 across a data network, such as the Internet 212.

In general terms, the client terminal 213 may be utilised by a hospital administration staff, surgeons and the like for the purposes of inputting, editing and viewing schedules and the like. Furthermore, the schedule server 214 may be a hospital server or the like comprising the patient procedure training data 101 which, in embodiments, may execute a calendar application such as one implemented by the Microsoft exchange server for the purposes of promoting a calendar having the schedule 106.

Furthermore, the optimising server 222 interfaces the client terminals 213 and the schedule server 214 by retrieving the schedule data 107 from the schedule server 214 for the purposes of generating the throughput optimise schedule 110 and serving webpages to the client terminal 213 accordingly.

It should be noted that the system 200 need not necessarily be limited to this particular computer architecture and other architectures may be applicable within the purposive scope of the embodiments described herein.

The optimising server 222 may take the form of a physical server, such as a rackmounted server or alternatively a virtualised server instance, such as which may be implemented virtually by a server virtualisation service such as Amazon web services (AWS).

As can be seen, the optimising server 222 comprises a processor 209 for processing digital data. In operable communication with the processor 209 across a data bus 28 is a memory device 223. The memory device 223 may take the form of short-term RAM memory or longer term hard drive data storage or a combination of both.

The memory device 223 comprises computer program code which is fetched by the processor 209 for execution.

In this regard, the memory device 223 is configured with various software modules for implementing the various computer processes described herein. Specifically, the memory device 223 may comprise an operating system 207 such as the Linux kernel or the like which is read by the processor 209 during the bootstrap phase and the subsequent execution of the processes described herein.

Furthermore, the memory 223 may comprise various web applications including a Web server application 205 such as the Apache™ Web server application. The Web server application 205 may act in unison with a hypertext preprocessor 204 such as the PHP™ hypertext preprocessor and a database server 206 such as the MySQL™ database server so as to be able to generate dynamic HTTP responses to the various web requests received from the one or more client terminals 217.

Further specifically, for implementing the specific processes described herein, the memory device 223 may comprise a plurality of software modules which have been logically divided into data model 201, controller 202 and view 203 as will be referenced in further detail below.

The optimising server 223 may further comprise a network interface 211 for sending and receiving data across the Internet 212.

Similarly, the client terminal 217 comprises a processor 209 in operable communication with a memory device 223 across a system bus 208. Further similarly, the client terminal 217 also comprises a network interface 211 for sending and receiving data across the Internet 212 including when communicating with the optimising server 222.

The client terminal 217 may take on differing forms within the purposive scope of the embodiments described herein including personal computing devices such as desktop, laptop devices or the like or mobile communication devices such as smart phones. Furthermore, the mobile communication device 217 may allow a user interaction with the optimising server 223 utilising a web browser application (such as the Mozilla Firefox or Google Chrome web browser application) or alternatively by way of a downloadable software application “app” which is downloaded and installed for execution on the client terminal 217 from an App Store, such as the Apple App Store.

In embodiments, the client terminal 213 may implement a scheduler application 215 such as a calendar application such as the Microsoft Outlook software application which may be updated utilising the throughput optimise schedule 110.

Furthermore, the client terminal 217 may comprise a display device 217 for the display of digital data and an I/O interface 219 for receiving user interface commands.

Further similarly, the schedule server 214 also comprises a network interface 211 for communicating across the Internet 212 and a processor 209 for processing digital data. Furthermore, the memory device 223 of the schedule server 214 may comprise the schedule 106 which may be fetched by the optimising server 222 for the purposes of optimising the schedule.

### Model View Controller

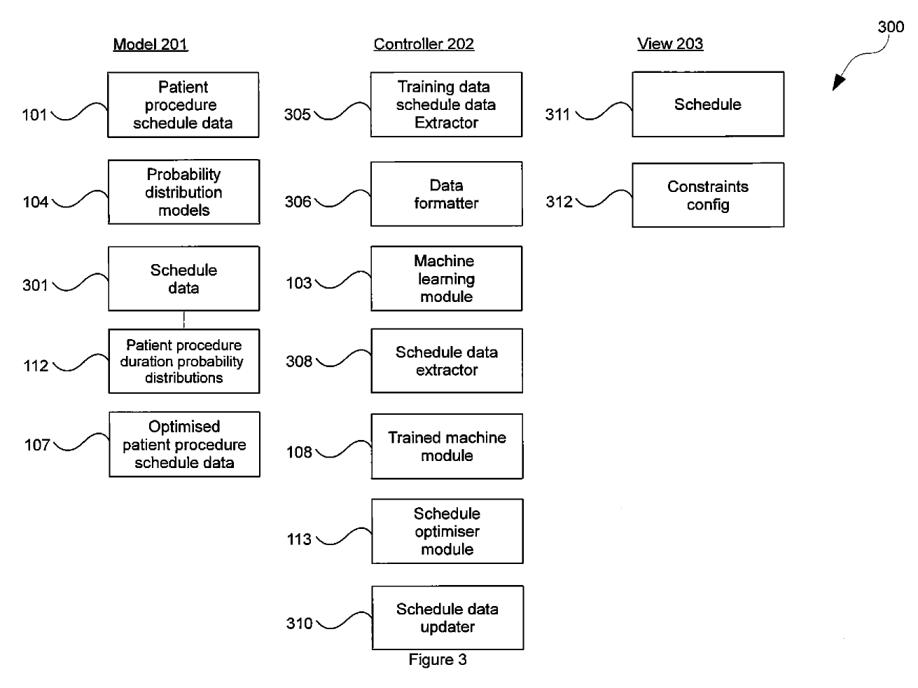

Having generally described the above system architecture, reference is now made to FIG. 3 wherein an exemplary data model View Controller (MVC) representation 300 of the present embodiments is provided for illustrative convenience.

As can be seen, the representation 100 comprises the data model 201, the controller 202 and the view 203 components.

In very general terms, the data model 201 represents the data and data structure, the view 202 represents the user interface and the controller 203 interfaces the user interface and the data model 101.

Considering initially the controller 202, the controller 202 comprises, in embodiments, a patient procedure training data extractor 305 which extracts the patient procedure training data 101, such as via the database interface 102.

The patient procedure training data 101 retrieved by the patient procedure training data extractor 305 may be stored as patient procedure training data 101 in the model 201.

Furthermore, the controller 202 may comprise a data formatter 306 to format the retrieved patient procedure training data 101 into the appropriate format including in the manner described in further detail below.

Furthermore, the machine learning module 103 controller may generate the probability distributions models 104 in the manner described above. The machine learning module 103 may store the calculated probability distributions models 104 within the model 201.

The controller 202 may further comprise a schedule data extractor 308 for the purposes of retrieving the schedule data 107 from the schedule server 214 or other schedule data source, including via the scheduler API 105.

Similarly, the schedule data extractor 308 may store the extracted schedule data as schedule data 301 within the model 201.

The controller 202 may further comprise the trained machine module 108 which is configured utilising the probability distribution models 104 so as to be able to take the schedule data 301 and generate the plurality of patient procedure duration probability distributions 112 for each of the proposed patient procedure schedules.

The controller 202 may further comprise the optimiser 113 to optimise to generate and store the optimise schedule data 107 in accordance with the probability distributions 303 and schedule data 301.

Furthermore, the controller 202 may comprise a schedule data updater 310 to update the schedule 106 to be a throughput optimised schedule 101 including via scheduler API 111.

As is also represented in FIG. 3, the view 203 may comprise a schedule view 311 allowing the user to view and edit the schedule 106 and throughput optimise schedule 110.

Furthermore, the view may comprise a constraint configuration view 312 allowing the user to configure the constraints 109.

### EXEMPLARY ILLUSTRATIVE EMBODIMENT

Having described the above technical architecture, there will now be provided an exemplary embodiment primarily for illustrative purposes. It should be noted that the embodiments provided hereunder are exemplary only and that no technical limitation should necessarily be imputed to all embodiments accordingly.

In this example embodiment, users, such as surgeons, hospital staff and the like may authenticate with a system 200 using the client terminal 213 so as to view, from the schedule view 311, a proposed schedule in accordance with his or her permission levels. For surgeons, the proposed schedule may comprise those sessions relating to the particular surgeon's specialty.

As will be described in further detail below, each session may be displayed with an associated risk characteristic being a session duration probability duration. In embodiments, the session duration probably duration may represent probabilities that the proposed session finishes:
a. More than 45 minutes early;
b. Between 45 minutes early and 15 minutes early;
c. Between 15 minutes early and 15 minutes late;
d. Between 15 minutes late and 45 minutes late; and
e. More than 45 minutes late

In embodiments, the probability categories may be configured by an administrator user.

Associated with each session may be patient specific information such as:
a. Name
b. Unique ID provided by the hospital
c. Procedure
d. Estimated procedure time
e. Surgical priority category, which may comprise:
   i. Category 1: Treat within 30 days
   ii. Category 2: Treat within 90 days
   iii. Category 3: Treat within 12 months
f. How overdue (or “underdue”) the patient is

Being presented with the schedule, the user may book the patient procedure sessions. Alternatively, should the user not be satisfied with the proposed sessions, such as due to factors external to the statistical optimisation, the user may make modifications to the session. In this embodiment, where the schedule sessions are modified, the system 200 is configured for calculating the risk metrics for the modified schedule allowing the user to make modifications to the schedule in confidence without making the schedule unnecessarily risky.

The training data extractor 305 extracts prior procedure data from a hospital database 101. Typically the patient procedure training data extractor 305 is provided with appropriate authentication credentials so as to authenticate with the relevant hospital databases so as to extract the data therefrom and modify it appropriately for utilisation by the system.

In embodiments, the patient procedure training data extractor 305 may comprise separate constituent modules comprising the extractor 305 and the formatter 306. The extractor obtains raw data from the hospital database and for inclusion in a set of files that the formatter 306 has access to.

The formatter 306 formats the raw data into a form suitable to be sent to the optimising server 223 (which may reside outside the hospital firewall). In a yet further embodiment, the formatter 306 also formats results received from the optimising server 223 into a form suitable for display to the end user.

Furthermore, the formatter 306 may perform a data preparation step before the data is sent to the machine learning module 103 wherein some patients on the waiting list have no surgeon assigned to them. For example, patients who require their tonsils to be removed can be treated by any Ear/Nose/Throat (ENT) surgeon. These surgeons are often recorded in the source data as: specialty=“ENT”, surgeon=“Any”. Now, the system 200 may assign these patients a surgeon such that their waiting time is minimised, after accounting for the case load of each ENT surgeon (waiting list length), the sessions the surgeons have scheduled (hours to be worked). This process occurs for other specialties as well. Also, other hospitals may request that this step be excluded from the pipeline between source data and optimised schedules.

In embodiments, the prior procedure data 101 comprises the name or ID of the surgeon performing the procedure, the procedure type and the duration of the procedure. In this manner, the system will be able to calculate statistical probabilities relating to particular types of procedures performed by particular surgeons. In embodiments, other information (including that which affects the statistical calculations) may be included within the prior procedure data 101 such as the name or ID of the patient, patient demographics (such as age, gender or the like), the date on which the procedure was performed or any other information relevant for the cockle elation of the probability distributions 104.

In embodiment, the throughput optimise schedule 110 as optimised by the trained machine module 108 may be displayed within the schedule view 311 as follows:
a. Scheduled Patients
   i. Contains proposed schedules.
   ii. Column names: Session ID, Date, Theatre, UR Number (patient id number), Patient Name, Surgeon, Specialty, Category, Overdue %, Booking Status, Estimated Duration, Procedure Description, Prob >45 mins early (%), Prob 15-45 mins early (%), Prob Within 15 mins (%), Prob 15-45 mins late (%), Prob >45 mins late (%).
b. Unplaced Patients
   i. Contains patients who are eligible to be scheduled but were not included due to a lack of available operating time.
   ii. Column names: UR Number, Patient Name, Surgeon, Specialty, Category, Overdue %, Estimated Duration, Procedure Description.
c. Patients with Data Issues.
   i. Contains patients who could not be scheduled due to either a data issue (e.g., missing estimated procedure duration), or not being ready for care until after the period being scheduled (typically 4 to 8 weeks).
   ii. Column names: UR Number, Patient Name, Surgeon, Specialty, Category, Overdue %, Estimated Duration, Procedure Description, Data Issue.
d. Empty Sessions.
   i. Contains sessions that remain empty after the optimisation has occurred. For example, a surgeon may have 2 sessions available, but a waiting list of only 2 patients. The patients are scheduled into one session, leaving the other session empty.
   ii. Column names: Session ID, Date, Theatre, Surgeon, Specialty.

The schedule data 107 input into the trained machine module 108 may comprise at least one of Surgeon ID; Patient ID; Procedure type; Estimated procedure duration; Surgical priority category and a patient due metric

For example, in one embodiment, the schedule data 107 may comprise patient identifying information and proposed procedure type. The associated surgeon performing the procedure may also be input. In embodiments, the surgeon may estimate the procedure time so as to assist the scheduler 11 in the optimisation. As will be described in further detail below, in embodiments, the trained machine module 108 may apply a weighting to the surgeons estimated procedure time depending on various factors, such as the extent and availability of prior procedure data 101.

In one embodiment, the procedure type is not sent to the machine learning module 103, (but is displayed in the 311 for user convenience) wherein the only input to the machine learning module 103 is surgeon ID, surgeon specialty, estimated procedure duration and prior durations for each of procedure+anaesthetic.

This prior data may extend back 12 months if available as the 12 months is an adequate balance between having enough data, and not using irrelevant data such that, a surgeon's performance may change over time as the surgeon becomes more experienced and/or takes on new kinds of cases. As such, taking too much data may result in inaccurate modelling.

The observed or recorded procedure time is required for the machine learning module 103 to construct the distribution of each case's anaesthetic+procedure duration. The prior procedure time may also used by the trained machine module 108 to construct candidate sessions, of which there are often billions. The optimiser module 113 then selects sessions from the candidates such that the probabilities are favourable and the constraints are respected.

In embodiments, additional constraints 109 may be input to the scheduler 11, such as the above-described surgical priority category and the patient due metric (such as overdue or underdue and duration).

Specifically, the trained machine module 108 may be configured for maximising patient throughput while respecting the following constraints:
a. Having good statistical properties (such as a good chance of finishing within 15 minutes of the session's scheduled finishing time);
b. Keeping booked patients in the session to which they have been assigned;
c. Ensuring patients are treated on or before their due date;
d. Prioritising patients who are already overdue, or who are becoming overdue in the next 4 weeks;
e. Ensuring patients are only proposed for sessions that they can attend;
f. Ensuring patients are proposed for a maximum of one 1 session; and
g. Ensuring a minimum amount of time per surgeon in sessions that are shared by multiple surgeons.

Additionally, the trained machine module 108 may take into account the Not Ready For Care (NRFC) status constraint of patients. Specifically, some patients have dates that they cannot attend for certain reasons such as work, school camp, sickness, etc. This information may be included in the input data and the trained machine module 108 ensures that patients are scheduled for dates that they can attend.

Additionally, the trained machine module 108 may take into account shared sessions shared by multiple surgeons. For example, three orthopedic surgeons may share a session every Monday in addition to conducting their own (non-shared) sessions on other days. In the Monday session each of the three surgeons conducts a case or two. That is, a given patient is not operated on by multiple surgeons; instead the session time is divided among surgeons.

Herein, the time allocated to each surgeon in a shared session varies and is currently not recorded. For example, currently, the three surgeons simply fill the Monday session with some cases each. It is not clear what factors influence the decision to include or exclude a patient from the session. As such, the system 200 uses the prior data 101 to estimate the proportion of the session that each surgeon should get, and then fills the session with these proportions in mind so as to bring consistency into the session division decision, rather than ad hoc reasoning.

The system 200 may calculate schedules up to 8 weeks in advance of the scheduled surgery date. Furthermore, as hospitals are dynamic environments and circumstances change daily (e.g. patients and staff get sick and become unavailable for a scheduled surgery; new patients with higher priority procedures arrive, and so on), the system 200 may be configured such that the proposed lists are automatically recalculated every morning at about 7 am, respecting the aforementioned constraints.

The statistical modelling performed by the machine learning module 103 will now be described in further detail below for further illustrative purposes to describe the functionality of the statistical machine learning module 103 in accordance with the various embodiments.

In embodiments, the machine learning module 103 takes as input a prior duration of a patient's procedure, and outputs the (modelled) distribution of the duration of: anaesthetic time+procedure time. Herein, the modelling of anaesthetic time accounts for variation in anaesthetists and the complexity of anaesthetic for different procedures. In other words, the anaesthetic is just as varied and complex as the surgery itself, just shorter.

The trained machine module 108, utilising the modelled probability distribution models 115 then calculates the probability distributions 112 for each of the schedules.

In embodiments, the optimiser module 113 may combines the distributions for individual sessions to arrive at a distribution for a whole session. The optimiser module 113 then searches through (almost) all possible sessions to find the ones with the best chance of finishing within 15 mins of the allocated time. At the same time, sessions with a greater than 20% chance of finishing more than 45 mins late are identified and excluded from consideration.

In embodiments, the machine learning module 103 may perform statistical modelling for each surgeon so as to account for different experience levels, estimation accuracy and procedure types among surgeons, despite similar estimated durations.

For example, the machine learning module 103 may produce a probability distribution for each estimated procedure duration for Surgeon A. As such, if Surgeon A estimates that a particular patient will take 60 mins (not including anaesthetic), then the model produces a distribution of the duration of anaesthetic+procedure for cases who are to be treated by Surgeon A and who are estimated by the surgeon to take 60 mins.

In embodiments, the machine learning module 103 is configured such that, if Surgeon A is new or otherwise has a short track record, and there is therefore not enough data to produce a reliable model, the machine learning module 103 uses a weighted average of the track records of the surgeons in the same specialty.

The weights used may depend on the prior data 13 available. Specifically, if Surgeon A has a long track record, then his/her weight will be almost 1, which means his/her track record is relied upon almost exclusively by the machine learning module 103. Conversely, if Surgeon A's track record is short, then the weight is decremented by the machine learning module 103 such that other surgeon track records for surgeons in the same specialty become more influential in producing the model for Surgeon A. For example, if there are 3 surgeons in Surgeon A's specialty, each with a short track record, then Surgeon A's model will be made up of all 3 surgeons' track records with approximately equal weight (⅓ each).

Furthermore, the machine learning module 103 may be configured to account for circumstances where there is little prior data 9 available for procedures having a particular duration estimation. For example, if Surgeon A has a procedure estimated to take 4 hours, and has a long track record, but a short track record for procedures estimated to take 4 hours, then the machine learning module 103 allocates more weight to data from other surgeons who have done 4 hour cases. Further, if Surgeon A has a long track record with cases estimated to take a similar duration, such as 3.5 hours, then the machine learning module 103 may apply a weighting to this prior data too.

### Interpretation

### Wireless:

The invention may be embodied using devices conforming to other network standards and for other applications, including, for example other WLAN standards and other wireless standards. Applications that can be accommodated include IEEE 802.11 wireless LANs and links, and wireless Ethernet.

In the context of this document, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In the context of this document, the term “wired” and its derivatives may be used to describe circuits, devices, systems, methods, techniques, communications channels, etc., that may communicate data through the use of modulated electromagnetic radiation through a solid medium. The term does not imply that the associated devices are coupled by electrically conductive wires.

### Processes:

Unless specifically stated otherwise, as apparent from the following discussions, it is appreciated that throughout the specification discussions utilizing terms such as “processing”, “computing”, “calculating”, “determining”, “analysing” or the like, refer to the action and/or processes of a computer or computing system, or similar electronic computing device, that manipulate and/or transform data represented as physical, such as electronic, quantities into other data similarly represented as physical quantities.

### Processor:

In a similar manner, the term “processor” may refer to any device or portion of a device that processes electronic data, e.g., from registers and/or memory to transform that electronic data into other electronic data that, e.g., may be stored in registers and/or memory. A “computer” or a “computing device” or a “computing machine” or a “computing platform” may include one or more processors.

The methodologies described herein are, in one embodiment, performable by one or more processors that accept computer-readable (also called machine-readable) code containing a set of instructions that when executed by one or more of the processors carry out at least one of the methods described herein. Any processor capable of executing a set of instructions (sequential or otherwise) that specify actions to be taken are included. Thus, one example is a typical processing system that includes one or more processors. The processing system further may include a memory subsystem including main RAM and/or a static RAM, and/or ROM.

### Computer-Readable Medium:

Furthermore, a computer-readable carrier medium may form, or be included in a computer program product. A computer program product can be stored on a computer usable carrier medium, the computer program product comprising a computer readable program means for causing a processor to perform a method as described herein.

### Networked or Multiple Processors:

In alternative embodiments, the one or more processors operate as a standalone device or may be connected, e.g., networked to other processor(s), in a networked deployment, the one or more processors may operate in the capacity of a server or a client machine in server-client network environment, or as a peer machine in a peer-to-peer or distributed network environment. The one or more processors may form a web appliance, a network router, switch or bridge, or any machine capable of executing a set of instructions (sequential or otherwise) that specify actions to be taken by that machine.

Note that while some diagram(s) only show(s) a single processor and a single memory that carries the computer-readable code, those in the art will understand that many of the components described above are included, but not explicitly shown or described in order not to obscure the inventive aspect. For example, while only a single machine is illustrated, the term “machine” shall also be taken to include any collection of machines that individually or jointly execute a set (or multiple sets) of instructions to perform any one or more of the methodologies discussed herein.

### Additional Embodiments

Thus, one embodiment of each of the methods described herein is in the form of a computer-readable carrier medium carrying a set of instructions, e.g., a computer program that are for execution on one or more processors. Thus, as will be appreciated by those skilled in the art, embodiments of the present invention may be embodied as a method, an apparatus such as a special purpose apparatus, an apparatus such as a data processing system, or a computer-readable carrier medium. The computer-readable carrier medium carries computer readable code including a set of instructions that when executed on one or more processors cause a processor or processors to implement a method. Accordingly, aspects of the present invention may take the form of a method, an entirely hardware embodiment, an entirely software embodiment or an embodiment combining software and hardware aspects. Furthermore, the present invention may take the form of carrier medium (e.g., a computer program product on a computer-readable storage medium) carrying computer-readable program code embodied in the medium.

### Carrier Medium:

The software may further be transmitted or received over a network via a network interface device. While the carrier medium is shown in an example embodiment to be a single medium, the term “carrier medium” should be taken to include a single medium or multiple media (e.g., a centralized or distributed database, and/or associated caches and servers) that store the one or more sets of instructions. The term “carrier medium” shall also be taken to include any medium that is capable of storing, encoding or carrying a set of instructions for execution by one or more of the processors and that cause the one or more processors to perform any one or more of the methodologies of the present invention. A carrier medium may take many forms, including but not limited to, non-volatile media, volatile media, and transmission media.

### Implementation:

It will be understood that the steps of methods discussed are performed in one embodiment by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

### Means For Carrying out a Method or Function

Furthermore, some of the embodiments are described herein as a method or combination of elements of a method that can be implemented by a processor of a processor device, computer system, or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

### Connected

Similarly, it is to be noticed that the term connected, when used in the claims, should not be interpreted as being limitative to direct connections only. Thus, the scope of the expression a device A connected to a device B should not be limited to devices or systems wherein an output of device A is directly connected to an input of device B. It means that there exists a path between an output of A and an input of B which may be a path including other devices or means. “Connected” may mean that two or more elements are either in direct physical or electrical contact, or that two or more elements are not in direct contact with each other but yet still co-operate or interact with each other.

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### SCOPE OF INVENTION

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

1. A patient procedure schedule throughput optimiser system comprising:
a data extractor module configured for receiving patient procedure training data from a patient procedure schedule data database,
a machine learning module having as input the patient procedure training data, the patient procedure training data representing a plurality of prior patient procedures and a duration for each of the prior patient procedures and wherein the machine learning module is configured for training using the patient procedure training data for generating a plurality of patient procedure duration probability distribution models;
a trained machine module configured in accordance with the patient procedure duration probability distribution models and having as input schedule data, the schedule data representing a plurality of future patient procedures, and wherein the trained machine module is configured for calculating patient procedure duration probability distributions for each of the future patient procedures;
a schedule optimiser module having as input the patient procedure duration probability distributions and wherein the schedule optimiser module is configured for calculating an optimised patient procedure schedule in accordance with the patient procedure duration probability distributions and wherein the schedule optimiser module is configured for optimising the number of sessions of the optimised patient procedure schedule; and
a schedule interface configured to display the optimised patient procedure schedule.

2. A system as claimed in claim 1, wherein the schedule optimiser module is configured for calculating the optimised patient procedure schedule using probability distribution variance threshold discrimination of each of the patient procedure duration probability distributions for the plurality of future patient procedures.

3. A system as claimed in claim 1, wherein:
the patient procedure training data further represents a procedure type for each of the prior patient procedures; and
the schedule data further represents a procedure type for each of the future patient procedures and wherein:
the machine learning module is configured for generating the session duration probability distribution models further in accordance with the procedure type for each of the prior patient procedures; and
the optimiser module is further configured for generating the throughput optimised schedule further in accordance with the procedure type for each of the future patient procedures.

4. A system as claimed in claim 1, wherein:
the patient procedure training data further represents patient specific information for each of the prior patient procedures; and
the input schedule data further represents patient specific information for each of the future patient procedures and wherein:
the machine learning module is configured for generating the session duration probability distribution models further in accordance with the patient specific information for each of the prior patient procedures; and
the optimiser module is further configured for calculating the optimised patient procedure schedule further in accordance with the patient specific information for each of the future patient procedures.

5. A system as claimed in claim 1, wherein:
the patient procedure training data further represents surgeon specific information for each of the prior patient procedures; and
the input schedule data further represents surgeon specific information for each of the future patient procedures and wherein:
the machine learning module is configured for generating the session duration probability distribution models further in accordance with the surgeon specific information for each of the prior patient procedures; and
the optimiser module is further configured for calculating the optimised patient procedure schedule further in accordance with the surgeon specific information for each of the future patient procedures.

6. A system as claimed in claim 5, wherein the surgeon specific information includes a surgeon specified session duration estimations.

7. A system as claimed in claim 6, wherein the trained machine module is configured for calculating the patient procedure duration probability distributions in accordance with the session duration estimations.

8. A system as claimed in claim 7, the trained machine module is configured for calculating the patient procedure duration probability distributions in accordance with a weighting of the session duration estimations.

9. A system as claimed in claim 8, wherein the weighting is between 0 and 1.

10. A system as claimed in claim 8, wherein the trained machine module is configured for calculating the number of sessions allocated to the surgeon in accordance with the patient procedure training data and calculate the weighting in accordance with the number of sessions.

11. A system as claimed in claim 1, wherein the schedule optimiser module is configured for calculating the optimised patient procedure schedule in accordance with constraints.

12. A system as claimed in claim 11, wherein the constraints include a constraint of maintaining patients to assigned session.

13. A system as claimed in claim 11, wherein the constraints include a constraint of assigning patients to schedules on or before a patient treatment due date.

14. A system as claimed in claim 11, wherein the constraints include a prioritising allocation of patients who are already overdue or who are becoming overdue.

15. A system as claimed in claim 11, wherein the constraints include a constraint of assigning patients according to patient attendance ability constraints.

16. A system as claimed in claim 11, wherein the constraints include a constraint of ensuring patients are allocated for a maximum of one session.

17. A system as claimed in claim 11, wherein the constraints include a constraint of allocating surgeons at least a minimum amount of time for sessions that are shared by multiple surgeons.

18. A system as claimed in claim 1, wherein:
the patient procedure training data further represents a procedure type, patient specific information and surgeon specific information for each of the prior patient procedures; and
the input schedule data further represents procedure type, patient specific information and surgeon specific information for each of the future patient procedures and wherein:
the machine learning module is configured for generating the session duration probability distribution models further in accordance with the procedure type, patient specific information and surgeon specific information for each of the prior patient procedures; and
the optimiser module is further configured for calculating the optimised patient procedure schedule further in accordance with the procedure type, patient specific information and surgeon specific information for each of the future patient procedures.
